(19) United States
(12) Patent Application Publication
(10) Pub. No.: US 2008/0085514 A1
(43) Pub. Date: Apr. 10, 2008

(54) Methods and devices for array synthesis

(76) Inventors: Bill J. Peck, Mountain View, CA (US); Eric Leproust, San Jose, CA (US); Lawrence J. DaQuino, Los Gatos, CA (US); Heather Brummel McCuen, San Jose, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/546,177

(22) Filed: Oct. 10, 2006

(51) Int. Cl. C12Q 1/68; C12M 3/00

(52) U.S. Cl. 435/6; 435/287.2; 427/2.11

(57) ABSTRACT

Aspects of the invention include methods of fabricating an array. In an embodiment of the invention, a dry gas is vertically directed onto a solid support surface prior to deposition of a fluid reagent on a surface of the support. Also provided are devices and systems for practicing the methods.

### BACKGROUND OF THE INVENTION

Biopolymeric arrays (such as nucleic acid, e.g., DNA or RNA, arrays), are known and are used, for example, as diagnostic or screening tools. Such arrays include regions of usually different biopolymers arranged in a predetermined configuration on a surface of a solid support, such as a glass slide. These regions (sometimes referred to as “features”) are positioned at respective locations (“addresses”) on the surface of the solid support. The arrays, when exposed to a sample, will exhibit an observed binding pattern depending on the probe molecules in the feature location and the target molecules present in the sample. This binding pattern can be detected upon interrogating the array, where the interrogation protocol employed depends on the labeling scheme that is used.

For example, in a nucleic acid array assay, all polynucleotide targets (for example, DNA) in a sample can be labeled with a fluorescent label, and the fluorescence pattern on the array accurately observed following exposure to the sample. Assuming that the different sequence polynucleotides were correctly deposited in accordance with the predetermined configuration, then the observed binding pattern will be indicative of the presence and/or concentration of one or more polynucleotide targets of the sample.

Arrays can be fabricated by depositing previously obtained biopolymers onto a substrate, or by in situ synthesis methods. In situ fabrication methods include those described in U.S. Pat. No. 6,180,351 and WO 98/41531, and the references cited therein.

### SUMMARY OF THE INVENTION

Aspects of the invention include methods of fabricating an array. In an embodiment of the invention, a solid support surface is vertically contacted by a dry gas prior to deposition of a fluid reagent on the surface of the support. Also provided are devices and systems for practicing the methods.

### BRIEF DESCRIPTION OF THE DRAWINGS

According to common practice, the various features of the drawings may not be drawn to-scale. Rather, the dimensions of the various features may be arbitrarily expanded or reduced for clarity. Included in the drawings are the following figures:

FIG. 1 illustrates a solid support (e.g., a substrate) carrying multiples arrays, such as may be fabricated by methods of the present invention.

FIG. 2 is an enlarged view of a portion of FIG. 1 showing multiple ideal spots or features.

FIG. 3 is an enlarged illustration of a portion of the substrate in FIG. 2.

FIG. 4 schematically represents of a pulse jet head assembly in accordance with the invention.

FIG. 5 schematically illustrates an array fabrication system according to an embodiment of the present invention.

FIG. 6 is a schematic representation of a system of the invention.

FIG. 7 is a test run print of a microarray fabricated and tested in accordance with the methods of the invention.

FIG. 8 is a test run print of a microarray fabricated and tested in accordance with the methods of the invention.

FIG. 9 is a test run print of a microarray fabricated and tested in accordance with conventional methods, for instance, with the use of an anhydrous chamber.

FIG. 10 is a graphic representation of the results of test runs on 25mer microarrays that were fabricated using one of four different protocols. Signal strength is indicated on the Y-axis and test run is indicated on the X-axis.

FIG. 11 is a graphic representation of the results of test runs on 45mer microarrays that were fabricated using one of four different protocols. Signal strength is indicated on the Y-axis and test run is indicated on the X-axis.

FIG. 12 is a graphic representation of the results of test runs on 60mer microarrays that were fabricated using one of four different protocols. Signal strength is indicated on the Y-axis and test run is indicated on the X-axis.

### DEFINITIONS

The term “polymer” means any compound that is made up of two or more monomeric units covalently bonded to each other, where the monomeric units may be the same or different, such that the polymer may be a homopolymer or a heteropolymer. Representative polymers include polypeptides, polysaccharides, nucleic acids and the like, where the polymers may be naturally occurring or synthetic.

The term “peptide” as used herein refers to any compound produced by amide formation between an α-carboxyl group of one amino acid and an α-amino group of another group.

The term “oligopeptide” as used herein refers to peptides with fewer than about 10 to 20 residues, i.e., amino acid monomeric units.

The term “polypeptide” as used herein refers to peptides with more than 10 to 20 residues.

The term “protein” as used herein refers to polypeptides of specific sequence of more than about 50 residues.

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single-stranded nucleotide multimers of from about 10 up to about 200 nucleotides in length, e.g., from about 25 to about 200 nt, including from about 50 to about 175 nt, e.g. 150 nt in length

The term “polynucleotide” as used herein refers to single- or double-stranded polymers composed of nucleotide monomers which may be greater than about 100 nucleotides in length.

The term “functionalization” as used herein relates to modification of a solid support to provide a plurality of functional groups on the solid support surface. By a “functionalized surface” as used herein is meant a surface that has been modified so that a plurality of functional groups is present thereon.

The term “array” encompasses the term “microarray” and refers to an ordered distribution of features on a solid support surface presented for binding to ligands such as polymers, polynucleotides, peptide nucleic acids and the like.

The terms “reactive site”, “reactive functional group” or “reactive group” refer to moieties on a monomer, polymer or solid support surface that may be used as the starting point in a synthetic organic process. These sites are contrasted to “inert” hydrophilic groups that could also be present on a substrate surface, e.g., hydrophilic sites associated with polyethylene glycol, a polyamide or the like.

The term “oligomer” is used herein to indicate a chemical entity that contains a plurality of monomers. As used herein, the terms “oligomer” and “polymer” are used interchangeably for smaller nucleic acids that are prepared using the functionalized solid supports in accordance with the invention, particularly in conjunction with combinatorial chemistry techniques. Examples of oligomers and polymers include polydeoxyribonucleotides (DNA), polyribonucleotides (RNA), other polynucleotides which are C-glycosides of a purine or pyrimidine base, polypeptides (proteins), polysaccharides (starches, or polysugars), and other chemical entities that contain repeating units of like chemical structure. In certain embodiments, oligomers range from about 2 to about 50 monomers, such as from about 2 to about 20, and including from about 3 to about 10 monomers.

The term “monomer” as used herein refers to a chemical entity that can be covalently linked to one or more other such entities to form a polymer. For instance, nucleotide “monomers” that have first and second sites (e.g., 5′ and 3′ sites) suitable for binding to other like monomers by means of standard chemical reactions (e.g., nucleophilic substitution), and a diverse element which distinguishes a particular monomer from a different monomer of the same type (e.g., a nucleotide base, etc.). Synthesis of nucleic acids of this type utilizes an initial solid support-bound monomer that is used as a building-block in a multi-step synthesis procedure to form a complete nucleic acid.

The term “ligand” as used herein refers to a moiety that is capable of covalently or otherwise chemically binding a compound of interest. The arrays of solid support-bound ligands produced by the methods in accordance with the invention can be used in screening or separation processes, or the like, to bind a component of interest in a sample. The term “ligand” in the context of the invention may or may not be an “oligomer” as defined above. However, the term “ligand” as used herein may also refer to a compound that is “pre-synthesized” or obtained commercially, and then attached to the solid support surface.

The term “sample” as used herein refers to a material or mixture of materials that may or may not be in fluid form, and contains one or more components of interest.

The terms “nucleoside” and “nucleotide” are intended to include those moieties which contain not only the known purine and pyrimidine bases, but also other heterocyclic (e.g., nitrogenous hetercyclic) bases that have been modified. Such modifications include, but are not limited to: methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

As used herein, the term “amino acid” is intended to include not only the L, D- and nonchiral forms of naturally occurring amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine), but also modified amino acids, amino acid analogs, and other chemical compounds which can be incorporated in conventional oligopeptide synthesis, e.g., 4-nitrophenylalanine, isoglutamic acid, isoglutamine, ε-nicotinoyl-lysine, isonipecotic acid, tetrahydroisoquinoleic acid, α-aminoisobutyric acid, sarcosine, citrulline, cysteic acid, t-butylglycine, t-butylalanine, phenylglycine, cyclohexylalanine, β-alanine, 4-aminobutyric acid, and the like.

A biomonomer fluid or biopolymer fluid references a liquid containing either a biomonomer or biopolymer, respectively (e.g., in solution).

A “phosphoramidite” includes a group of the structure of formula (I) below:

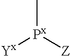

wherein either X is a linking atom such as O or S and may be the same or different; Y is a protecting group such as cyanoethyl; Z may be a halogen (particularly Cl or Br) or a secondary amino group such as morpholino or N(lower alkyl)2 where the alkyl groups are the same or different, for instance, N(i-propyl)2. By “lower alkyl” is referenced 1 to 8 C atoms.

A “nucleoside phosphoramidite” has a nucleoside or a nucleoside analog with the sugar ring bonded to the free bond on the X in formula (I). For example, one particular nucleoside phosphoramidite is represented by formula (II) below:

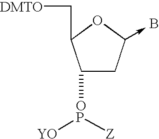

wherein B is a nucleoside base, and DMT is dimethoxytrityl. The O (which may instead be replaced by S) to which DMT is bonded, acts as a second linking group which is protected by the DMT. Protecting groups other than DMT may be used, and their removal during deprotection is known in oligonucleotide synthesis. Other nucleoside phosphoramidites are also known, for example ones in which the phosphoramidite group is bonded to a different location on the 5-membered sugar ring. Phosphoramidites and nucleoside phosphoramidites are described in U.S. Pat. No. 5,902,878, U.S. Pat. No. 5,700,919, U.S. Pat. No. 4,415,732, PCT publication WO 98/41531 and the references cited therein (the disclosures of which are herein incorporated by reference), among others.

A “group” includes both substituted and unsubstituted forms. It will also be appreciated that throughout the present application, words such as “upper”, “lower” and the like are used with reference to a particular orientation of the apparatus with respect to gravity, but it will be understood that other operating orientations of the apparatus or any of its components, with respect to gravity, are possible.

Reference to a “droplet” being dispensed from a pulse-jet herein, merely refers to a discrete small quantity of fluid (usually less than about 1000 pL) being dispensed upon a single pulse of the pulse-jet (corresponding to a single activation of an ejector) and does not require any particular shape of this discrete quantity. When a “spot” is referred to, this may reference a dried spot on the substrate resulting from drying of a dispensed droplet, or a wet spot on the substrate resulting from a dispensed droplet which has not yet dried, depending upon the context.

“Fluid” is used herein in its conventional sense to denote either a gaseous or liquid phase.

Use of the singular in reference to an item, includes the possibility that there may be multiple numbers of that item.

The term “protecting group” refers to chemical moieties that, while stable to the reaction conditions, mask or prevent a reactive group from participating in a chemical reaction. Protecting groups may also alter the physical properties such as the solubility of compounds, so as to enable the compounds to participate in a chemical reaction. Examples of protecting groups may be those described in: Greene et al., Protective Groups in Organic Synthesis, 2nd Ed., New York: John Wiley & Sons, 1991.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

The term “array” refers to any one-dimensional, two-dimensional or substantially two-dimensional (as well as a three-dimensional) arrangement of addressable regions (i.e., features) bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region.

As such, an “addressable array” includes any one or two or even three-dimensional arrangement of discrete regions (or “features”) bearing particular biopolymer moieties (for example, different polynucleotide sequences) associated with that region and positioned at particular predetermined locations on the substrate (each such location being an “address”). These regions may or may not be separated by intervening spaces. Arrays of interest include arrays of polymeric binding agents, where the polymeric binding agents may be any of: polypeptides, proteins, nucleic acids, polysaccharides, synthetic mimetics of such biopolymeric binding agents, etc. In certain embodiments, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, for instance, attached at one of their termini (e.g. the 3′ or 5′ terminus). Sometimes, the arrays are arrays of polypeptides, e.g., proteins or fragments thereof.

Any given solid support may carry one, two, four or more arrays disposed on a front surface of the substrate. Depending upon the intended use, any or all of the arrays may be the same or different from one another and each may contain multiple spots or features. An array may contain more than ten, more than one hundred, more than one thousand or more than ten thousand features, or even more than one hundred thousand features, in an area of less than 20 cm² or even less than 10 cm². Features may have widths (that is, diameter, for a round spot) in the range from about 10 μm to 1.0 cm. In other embodiments each feature may have a width in the range of about 1.0 μm to about 1.0 mm, such as from about 5.0 μm to about 500 μm, and including from about 10 μm to about 200 μm. Non-round features may have area ranges equivalent to that of circular features with the foregoing width (diameter) ranges.

In certain embodiments, at least some, or all, of the features are of different compositions (for example, when any repeats of each feature composition are excluded the remaining features may account for at least 5%, 10%, or 20% of the total number of features). Interfeature areas may be present which do not carry any ligand. Such interfeature areas may be present where the arrays are formed by processes involving drop deposition of reagents but may not be present when, for example, light directed synthesis fabrication processes are used. It will be appreciated though, that the interfeature areas, when present, could be of various sizes and configurations.

Each array may cover an area of less than about 100 cm², or even less than about 50 cm², about 10 cm² or about 1 cm². In certain embodiments, the solid support carrying the one or more arrays is shaped as a rectangular solid (although other shapes are possible), having a length of more than about 4 mm and less than about 1 m, such as more than about 4 mm and less than about 600 mm, including less than about 400 mm; a width of more than about 4 mm and less than about 1 m, such as about 500 mm, e.g., less than about 400 mm; and a thickness of more than about 0.01 mm and less than 5.0 mm, such as more than about 0.1 mm, e.g. less than about 2 mm, including more than about 0.2 and less than about 1 mm. With arrays that are read by detecting fluorescence, the solid support may be of a material that emits low fluorescence upon illumination with the excitation light. Additionally in this situation, the solid support may be relatively transparent to reduce the absorption of the incident illuminating laser light and subsequent heating if the focused laser beam travels too slowly over a region. For example, a solid support may transmit at least 20%, or 50% (or even at least 70%, 90%, or 95%), of the illuminating light incident on the front surface as may be measured across the entire integrated spectrum of such illuminating light or alternatively at 532 nm or 633 nm.

As is described in greater detail herein below, arrays may be fabricated using drop deposition from the modified pulse jet heads of the invention. The arrays may be fabricated of either precursor units (such as nucleotide or amino acid monomers) in the case of in situ fabrication, or of a previously obtained biomolecule, e.g., polynucleotide or polypeptide. Such methods are described below and may include aspects of the methods described in, for example, U.S. Pat. Nos. 6,242,266; 6,232,072; 6,180,351; 6,171,797; and 6,323,043; as well as U.S. patent application Ser. No. 09/302,898 and the references cited therein, with the appropriate modifications being made to the fluid dispensing head (e.g., pulse jet) assemblies and their methods of use, in accordance with the teachings of the instant invention.

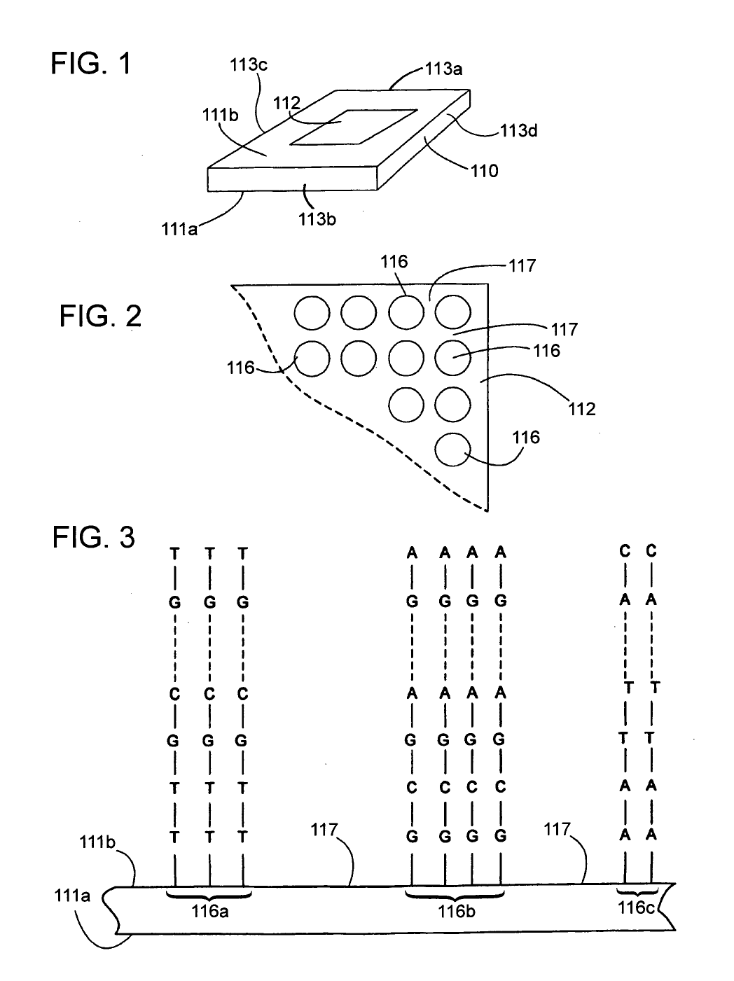

An exemplary chemical array is shown in FIGS. 1-3, where the array shown in this embodiment includes a contiguous planar solid support (also referred to herein as a substrate) 110 carrying an array 112 disposed on a rear surface 111b of support 110. It will be appreciated though, that more than one array (any of which are the same or different) may be present on rear surface 111b, with or without spacing between such arrays. That is, any given support may carry one, two, four or more arrays disposed on a surface of the support and depending on the use of the array, any or all of the arrays may be the same or different from one another and each may contain multiple spots or features. The one or more arrays 112 usually cover only a portion of the rear surface 111b, with regions of the rear surface 111b adjacent the opposed sides 113c, 113d and leading end 113a and trailing end 113b of slide 110, not being covered by any array 112. A front surface 111a of the slide 110 does not carry any arrays 112. Array 112 can be designed for testing against any type of sample, whether a trial sample, reference sample, a combination of them, or a known mixture of biopolymers, such as polynucleotides. Support 110 may be of any shape, as mentioned above.

As mentioned above, array 112 contains multiple spots or features 116 of biopolymers, e.g., in the form of polynucleotides. As mentioned above, all of the features 116 may be different, or some or all could be the same. The interfeature areas 117 could be of various sizes and configurations. Each feature carries a predetermined biopolymer, such as a predetermined polynucleotide (which includes the possibility of mixtures of polynucleotides). It will be understood that there may be a linker molecule (not shown) of any known types between the rear surface 111b and the first nucleotide.

Solid support 110 may carry on front surface 111a, an identification code, e.g., in the form of bar code (not shown) or the like printed on a support in the form of a paper label attached by adhesive or any convenient means. The identification code contains information relating to array 112, where such information may include, but is not limited to, an identification of array 112, i.e., layout information relating to the array(s), etc. The support may be porous or non-porous. The support may have a planar or non-planar surface.

In those embodiments where an array includes two or more features immobilized on the same surface of a solid support, the array may be referred to as addressable. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are may be separated by intervening spaces. In the case of an array, the “target” will be referenced as a moiety in a mobile phase (e.g., fluid), to be detected by probes (“target probes”) which are bound to the substrate at the various regions. However, either of the “target” or “probe” may be the one which is to be evaluated by the other (thus, either one could be an unknown mixture of analytes, e.g., polynucleotides, to be evaluated by binding with the other).

An array “assembly” includes a solid support and at least one chemical array, e.g., on a surface thereof. Array assemblies may include one or more chemical arrays present on a surface of a device that includes a pedestal supporting a plurality of prongs, e.g., one or more chemical arrays present on a surface of one or more prongs of such a device. An assembly may include other features (such as a housing with a chamber from which the substrate sections can be removed). “Array unit” may be used interchangeably with “array assembly”. “Hybridizing” and “binding”, with respect to polynucleotides, are used interchangeably.

The term “solid support” as used herein refers to a surface upon which marker molecules or probes, e.g., an array, may be adhered. A solid support may be configured as a substrate. Glass slides are the most common substrate for biochips, although fused silica, silicon, plastic and other materials are also suitable.

When two items are “associated” with one another they are provided in such a way that it is apparent one is related to the other such as where one references the other. For example, an array identifier can be associated with an array by being on the array assembly (such as on the substrate or a housing) that carries the array or on or in a package or kit carrying the array assembly. “Stably attached” or “stably associated with” means an item's position remains substantially constant where in certain embodiments it may mean that an item's position remains substantially constant and known.

“Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

“Rigid” refers to a material or structure which is not flexible, and is constructed such that a segment about 2.5 by 7.5 cm retains its shape and cannot be bent along any direction more than 60 degrees (and often not more than 40, 20, 10, or 5 degrees) without breaking.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

“Stringent hybridization conditions” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions sets forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant about 5-fold more, such as less than about 3-fold more. Other stringent hybridization conditions may also be employed, as appropriate.

“Contacting” means to bring or put together. As such, a first item is contacted with a second item when the two items are brought or put together, e.g., by touching them to each other.

“Depositing” or “dispensing” means to position (i.e., place) an item at a location or otherwise cause an item to be so positioned or placed at a location. Depositing includes contacting one item with another. Depositing or dispensing may be manual or automatic, e.g., “depositing” an item at a location may be accomplished by automated robotic devices. As used herein, “depositing a solid activator” at a location encompasses depositing or dispensing a fluid composition comprising activator at a location and removing fluid from the composition so that the solid activator remains.

By “remote location,” it is meant a location other than the location at which the array (or referenced item) is present and hybridization occurs (in the case of hybridization reactions). For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart.

“Communicating” information means transmitting the data representing that information as signals (e.g., electrical, optical, radio signals, and the like) over a suitable-communication channel (for example, a private or public network).

“Forwarding” an item refers to any means of getting that item from one location to the next, whether by physically transporting that item or otherwise (where that is possible) and includes, at least in the case of data, physically transporting a medium carrying the data or communicating the data.

An array “package” may be the array plus only a substrate on which the array is deposited, although the package may include other features (such as a housing with a chamber).

A “chamber” references an enclosed volume (although a chamber may be accessible through one or more ports). It will also be appreciated that throughout the present application, that words such as “top,” “upper,” and “lower” are used in a relative sense only.

It will also be appreciated that throughout the present application, that words such as “cover”, “base” “front”, “back”, “top”, are used in a relative sense only. The word “above” used to describe the substrate and/or flow cell is meant with respect to the horizontal plane of the environment, e.g., the room, in which the substrate and/or flow cell is present, e.g., the ground or floor of such a room.

### DETAILED DESCRIPTION

Aspects of the invention include methods of fabricating an array. In an embodiment of the invention, a dry gas is vertically directed onto a solid support surface prior to deposition of a fluid reagent onto the surface of the support. In other words, a dry gas is vertically contacted with a solid support surface. Also provided are devices and systems for practicing the methods.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing aspects of the invention, methods in accordance with the invention are reviewed first in greater detail, followed by a discussion of various systems and components thereof that may be used in the methods of the invention.

### Methods for Fabricating an Array of Biopolymers

As summarized above, the subject invention provides methods for fabricating an array of biopolymers on a surface of a solid support (e.g., a substrate). Specifically, an aspect of the invention is a method for depositing a reagent fluid onto a surface of a solid support. In the subject methods, a gas is first directed substantially vertically onto a region of a surface of a solid support. The contact of the gas with the solid support produces a dried region. Once a dried region is produced, a reagent fluid is dispensed onto the dried region.

Accordingly, in practicing the subject methods, a fluid dispensing head assembly, such as a print jet head assembly, may be used to deliver both a gas and a fluid reagent to the surface of a solid support. Hence, as will be described in greater detail herein below, the fluid dispensing head assembly includes both a gas jet and a pulse jet. The subject methods, therefore, may include positioning a pulse jet head assembly containing at least one gas jet and at least one pulse jet in proximity to the surface of a solid support. A gas is then directed substantially vertically from a gas jet toward the surface in a manner sufficient to contact the gas with the surface of the solid support so as to produce a dried region on the surface. Next, a reagent fluid is dispensed from a pulse jet onto the dried region on the surface of the solid support.

Any suitable solid support may be used. Accordingly, a suitable solid support may have a variety of forms and compositions and can be derived from naturally occurring materials, naturally occurring materials that have been synthetically modified, or synthetic materials. Examples of suitable support materials include, but are not limited to, glasses, controlled pore glass (CPG”), cellulosic polymers, nitrocellulose, polyacrylamides, quartz, silicon or silicon covered with silicon dioxide, ceramics, silicas, teflons, and metals (for example, gold, platinum, and the like). Suitable materials also include polymeric materials, including plastics (for example, polytetrafluoroethylene, polypropylene, polystyrene, polycarbonate, and blends thereof, and the like), polysaccharides such as agarose (e.g., that are available commercially as Sepharose®, from Pharmacia) and dextran (e.g., those available commercially under the tradenames Sephadex® and Sephacyl®, also from Pharmacia), polyacrylamides, polystyrenes, polyvinyl alcohols, copolymers of hydroxyethyl methacrylate and methyl methacrylate, and the like. A solid support may be obtained commercially and used as is, or may be treated or coated prior to use.

The surface of the solid support may be substantially planar, although planarity is not required and the surfaces can be of any geometry suitable for contact with fluid reagents used in the formation of an array. For instance, the surface of the solid support onto which the biopolymer or monomeric precursor thereof is bound can be smooth and planar, or have irregularities, such as depressions or elevations thereon. The configuration of the support can be selected according to manufacturing, handling, and use considerations.

Accordingly, the solid support may have any of a variety of configurations ranging from simple to complex. In certain embodiments, the solid support may have a planar form, as for example a slide or plate configuration, such as a rectangular, square or disc configuration. Accordingly, in certain embodiments, the solid support may be shaped as a rectangular solid substrate, having a length in the range of about 4 mm to about 300 mm, including about 4 mm to about 150 mm, as well as about 4 mm to about 125 mm. In certain embodiments, the solid support may have a width in the range of about 4 mm to about 300 mm, including about 4 mm to about 120 mm, as well as about 4 mm to about 80 mm. In certain embodiments, the solid support may have a thickness in the range of about 0.01 mm to about 5.0 mm, including from about 0.1 mm to about 2 mm, as well as about 0.2 to about 1 mm. In certain embodiments, the solid support may be shaped as a wafer. As will be described in greater detail below, an aspect of the invention allows for the fabrication of a biopolymer array on the surface of a solid support without the employment of a coupling chamber. Accordingly, a device of the invention allows for the use of a larger solid support than is often employed in the art, without a corresponding scaling of an anhydrous chamber.

In certain embodiments, the surface of the solid support may be chemically modified (e.g., functionalized) with a surface energy modification reagent. For instance, in one embodiment, the surface of a solid support (e.g., a substrate) may first be functionalized by being contacted with a surface energy modification reagent, such as a silane-derivatizing composition that contains one or more types of silanes, which functionalizes the surface. Once the surface has been functionalized it may then be modified, e.g., via pulse-jet deposition of biopolymers or precursor residues thereof, so as to produce a surface with at least one feature location.

Specifically, in certain embodiments, the surface energy modification reagent is a silane-derivatizing composition. Accordingly, in certain embodiments, prior to deposition of a fluid reagent on the surface of the solid support, the surface may be derivatized by being contacted with a silane-derivatizing composition of one or more silanizing reagents. The derivatizing composition may include two or more types of silanes, which may be the same or different from one another. For instance, the two or more silanes may differ with respect to their leaving group substituents, which may include, but are not limited to: halogens, chloro, alkoxy, aryloxy moieties, lower alkyl, e.g., methyl, ethyl, isopropyl, n-propyl, t-butyl moieties, and the like. In certain embodiments, where a mixture of silanes make up the derivatizing composition, the first silane may be a derivatizing agent that reduces surface energy, while the second silane may provide a desired functionality.

Dispensing a Biopolymer or Monomeric Precursor Thereof onto the Surface of a Solid Support

As set forth above, an aspect of the invention is a method for depositing a reagent fluid onto a surface of a solid support. In accordance with the methods of the invention, a fluid dispensing head assembly, for instance, a pulse jet head assembly containing a gas jet apparatus (e.g., a gas jet) and a pulse jet apparatus (e.g., a pulse jet) is positioned in proximity to a surface of a solid support. By “in proximity to” is meant that the pulse jet head is positioned from about 0.001 mm to about 100 mm, for instance, about 0.1 mm to about 10 mm, including about 0.5 mm to about 2.5 mm, e.g., about 0.5 mm to about 1.5 mm, such as 1 mm, from the surface of the solid support.

Once the pulse jet head assembly is positioned in proximity to a surface of a suitable solid support, a dispensing sequence is then initiated for the fabrication of a chemical array of biopolymers on to the surface of the solid support. The dispensing sequence includes directing a gas substantially vertically from the gas jet assembly toward the surface of the solid support in a manner sufficient to contact the surface of the solid support with the gas.

By “substantially vertically” is meant that the gas exits the gas jet assembly in such a manner that its angle of contact with the solid support is greater than about 45°, for instance, greater than about 70°, including about 90°, wherein the surface of the solid support represents the X-Y plane. In certain embodiments, the gas is directed vertically in such a manner that its contact with the surface of the solid support is substantially normal to the surface of the solid support. By substantially “normal” is meant within about 50 of 90° (e.g., perpendicular to the surface of the solid support). For instance, in certain embodiments, “substantially normal” means that the gas is not directed onto the surface in a manner such that the gas flows parallel to the surface, i.e., horizontally across the surface, prior to contact with the surface.

Accordingly, an aspect of the invention is that the vertically directed gas contacts the surface of the solid support at a defined location or feature on the surface of the solid support and thereby substantially dries the area of contact (e.g., the feature location). The area of contact includes the region to which a biopolymer is to be added, e.g., the region of synthesis. By “substantially dries” is meant that the gas, for instance, an anhydrous gas, contacts the surface of the solid support in a manner sufficient to evaporate any moisture on the surface of the solid support to an extent that any moisture present does not affect the coupling reaction between the reactants (e.g., between the surface of the solid support and the biopolymer to be attached thereto).

The drying effect resulting from the gas contacting the surface of the solid support occurs rapidly. By “rapidly” is meant that the region of contact is dried within the time it takes for the feature location (e.g., area of fabrication) to move relative to the pulse jet head assembly, horizontally from the position wherein the gas jet is placed over the feature location to the position wherein the pulse jet is placed over the feature location. For instance, this time period may vary dependant on the stage speed, head assembly speed, and the like, but may be from about 0.01 seconds to about 10 seconds, including about 0.1 to about 5 seconds, for instance, about 1 second.

Once a desired feature location on the surface of the solid support has been substantially dried by being contacted with the dry gas, a composition (e.g., a reagent fluid) may then be dispensed from the pulse jet assembly in a manner sufficient to contact the dried feature location with the dispensed composition. Accordingly, the compositions of the invention may be deposited as droplets at a defined location or address using, for example, a pulse jet printing system that has been modified to deliver a flow of gas from the pulse jet head assembly to the surface of the solid support prior to and/or after the depositing of the droplet.

A composition of the invention may be a reagent fluid. The reagent fluid may be any suitable fluid that may be contacted and coupled to the surface of the solid support so as to facilitate the fabrication of an array. In certain embodiments, the composition may include a nucleotide monomer (or functionalized derivative thereof, a peptide monomer, an oligonucleotide, a polypeptide, a nucleic acid (e.g., a DNA, RNA, etc.), a protein fragment, a protein, a sugar, or the like. In certain embodiments, the composition may be a fluid activator composition, which may include a solvent.

In accordance with the methods described herein, an array can be fabricated by in situ synthesis methods (e.g., as are described below) or by depositing previously obtained reagents (e.g., nucleic acids, proteins, etc.) onto the surface of a solid support. For instance, a polynucleotide or polypeptide can be produced at a feature (e.g., a particular addressable position) on the surface of a solid support by sequentially depositing quantities of fluids (e.g., selected from four different solutions each containing a different nucleotide for polynucleotide synthesis) at the feature in a specific order in an iterative process so as to produce the polynucleotide or polypeptide. Alternatively, a preformed biopolymer, such as a nucleic acid or a protein, can be deposited directly at feature locations.

Accordingly, in certain embodiments, an aspect of the invention includes the fabrication of an array by in situ synthesis methods. In accordance with the methods of the invention, a polynucleotide or polypeptide can be fabricated in situ on the surface of a solid support by a) directing a gas vertically from an orifice of a gas jet assembly in a direction that is substantially normal to a feature location on the surface of the support where a biopolymer precursor is to be deposited and then b) depositing the biopolymer precursor onto the feature location on the surface of the solid support. As will be described below, in such an in situ synthesis method, this process is repeated numerous times for the fabrication of a polynucleotide or polypeptide array. Suitable in situ fabrication methods that may be modified in accordance with the teachings of the invention for use in the fabrication of biopolymer arrays include those described in U.S. Pat. No. 5,449,754 for synthesizing peptide arrays, and in U.S. Pat. No. 6,180,351 and WO 98/41531 for synthesizing polynucleotide arrays, the disclosures of which are incorporated herein by reference. It is to be noted, that although the following methods are described with reference to the production of a polynucleotide and/or nucleic acid, the methods herein disclosed can be used for the fabrication of a polypeptide or protein (e.g., a polypeptide array), with the appropriate modifications being made.

Specifically, a representative in situ method for fabricating a polynucleotide array follows the same iterative sequence used in forming one or more polynucleotides on a support, wherein the same process is repeated at each of the multiple different addresses at which features are to be formed. In certain embodiments, the sequence used to prepare a nucleic acid, e.g., oligonucleotide, using reagents of the type of formula (I) below, basically-follows the following steps.

First, the surface of a solid support is contacted at one or more feature locations, in a first iteration, with a dry gas, such as nitrogen, so as to substantially dry the feature location(s). In subsequent iterations, a deposited monomer unit(s) (e.g., of a growing polynucleotide or polypeptide sequence) is contacted with the dry gas.

Optionally the dried feature location(s) may then be contacted with a selected activator reagent. The activator reagent may be deposited so as to be positioned in the dried feature location(s) such that the activator substantially if not completely covers the feature location but little, if any, activator is present in any non-feature locations. Upon contact with a fluid monomer, the activator activates the first linking group of the monomer present in the fluid monomer (e.g., a phosphoramidite group as found in monomers employed in in situ nucleic acid production) such that the activated group will then link with a solid support or support bound moiety, e.g., a hydroxyl moiety (e.g., that is present on a silane-derivatized surface if it is the first residue or on a previously deposited residue), to produce a covalent bond, such that the monomer in the deposited second volume becomes covalently bound to the substrate surface, either directly or through one or more intervening monomeric residues of a polymeric ligand.

Once the activator reagent is contacted with the surface of the solid support at one or more feature locations, the feature location(s) containing the deposited activator reagent may be contacted with a dry gas so as to substantially dry the feature location(s).

Once the feature location is dried, a selected protected monomer, such as a nucleoside reagent (e.g., phosphoramidite), may be coupled through a phosphite linkage to the dry feature location on the support in the first iteration (which may contain an activator agent), or a previously deposited deprotected monomer (e.g., nucleoside) bound to the solid support in subsequent iterations, so as to produce a solid support containing at least one nucleoside reagent coupled to the surface thereof.

Next, any unreacted hydroxyl groups on the surface bound nucleoside(s) may be blocked and the phosphite linkage may be oxidized to form a phosphate linkage. Once the nucleoside reagent has been coupled to the solid support (in a first iteration) or a previously deposited deprotected monomer, the protecting group (“deprotection”) from the newly bound coupled nucleoside may be removed to generate a reactive site for the next cycle in which these steps are repeated (e.g., for the addition of another protected monomer for linking). Final deprotection of nucleoside bases can be accomplished using alkaline conditions such as ammonium hydroxide.

Accordingly, the functionalized support (in the first cycle) or deprotected coupled nucleoside (in subsequent cycles) provides a support bound moiety with a linking group that can be dried, activated and used for forming the phosphite linkage with the next nucleoside to be coupled after the drying step described above. The above steps of contacting with a dry gas, activator deposition and fluid monomer deposition can be repeated at each desired feature region on the solid support until an array of the desired biopolymers in the desired configuration has been synthesized. Accordingly, different monomers may be deposited at different regions on the solid support during any one cycle so that the different regions of a completed array will carry the different biopolymer sequences as desired in the completed array.

One or more intermediate further steps may be required in each iteration, such as oxidation and washing steps. Hence, it is understood, that intermediate drying, oxidation, deprotection, washing, activating and other steps may be performed between cycles. It is also to be noted that at any point in time in the iterative process a location of the solid support may be contacted with a dry gas, in accordance with the methods disclosed herein, so as to dry the specified location. These cycles may be repeated using different or the same monomers at multiple regions over multiple cycles as required to fabricate the desired array or arrays on a substrate. Following synthesis, the biopolymer can be cleaved from the solid support.

It is to be noted that although the above has been described with reference to a particular sequence of events, certain steps may be omitted or rearranged without substantially affecting the synthesis outcome. The methods of preparing polynucleotides are described in detail, for example, in Caruthers, Science 230: 281-285, 1985; Itakura et al., Ann. Rev. Biochem. 53: 323-356; Hunkapillar et al., Nature 310: 105-110, 1984; and in “Synthesis of Oligonucleotide Derivatives in Design and Targeted Reaction of Oligonucleotide Derivatives, CRC Press, Boca Raton, Fla., pages 100 et seq., U.S. Pat. No. 4,458,066, U.S. Pat. No. 4,500,707, U.S. Pat. No. 5,153,319, U.S. Pat. No. 5,869,643, EP 0294196, all of which can be modified for use in the methods of the present invention and are therefore incorporated herein by reference. Modified phosphoramidite and phosphite triester approaches are most broadly used, but other approaches which may be modified and employed include, but are not limited to, the phosphodiester approach, the phosphotriester approach and the H-phosphonate approach.

In situ polynucleotide synthesis methods, as described above, may use a nucleoside reagent (e.g., probe precursor) of the formula:

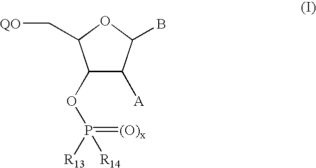

in which: A represents H, alkyl, or another substituent which does not interfere in the coupling of compounds of formula (I) to form polynucleotides according to the in situ fabrication process; B is a purine or pyrimidine base whose exocyclic amine functional group is optionally protected; Q is a conventional protective group for the 5′-OH functional group; x=0 or 1 provided:

(a) When x=1: R₁₃ represents H and R₁₄ represents a negatively charged oxygen atom; or R₁₃ is an oxygen atom and R₁₄ represents either an oxygen atom or an oxygen atom carrying a protecting group; and when x=0, R₁₃ is an oxygen atom carrying a protecting group and R₁₄ is either a hydrogen or a di-substituted amine group.

(b) When x is equal to 1, R₁₃ is an oxygen atom and R₁₄ is an oxygen atom, the method is in this case the so-called phosphodiester method; when R₁₄ is an oxygen atom carrying a protecting group, the method is in this case the so-called phosphotriester method.

(c) When x is equal to 1, R₁₃ is a hydrogen atom and R₁₄ is a negatively charged oxygen atom, the method is known as the H-phosphonate method.

(d) When x is equal to 0, R₁₃ is an oxygen atom carrying a protecting group and R₁₄ is either a halogen, the method is known as the phosphite method and; when x=0, R₁₃ is an oxygen atom carrying a protecting group, and R₁₄ is a leaving group of the disubstituted amine type, the method is known as the phosphoramidite method.

The amount and concentrations of the reagents employed in each synthesis step in the methods of the present invention is dependent on the nature of the reagents, solubility of the reagents, reactivity of the reagents, availability of the reagents, purity of the reagents, and so forth. The amounts and concentrations of the reagents are those necessary to achieve the overall synthesis of the chemical compound in accordance with the present invention. For instance, stoichiometric amounts may be employed, but excess of one reagent over the other may be used where circumstances dictate. Additionally, the concentration of nucleic acid precursor in the reagent fluid composition may be one that is sufficient to provide for the desired coupling during in situ synthesis. The time period for conducting the present method is dependent upon the specific reaction and reagents being utilized and the chemical compound being synthesized.

Embodiments of the methods lend themselves to synthesis of polynucleotides on array substrates in either the 3′-to-5′ or the 5′-to-3′ direction. In the former case, the synthesis process involves attachment of the initial nucleotide to a gas dried feature of the support at the 3′ position, leaving the 5′ position available for covalent binding of a subsequent monomer. In the latter case, the synthesis process involves attachment of an initial nucleotide to a gas dried feature of the support at the 5′ position, leaving the 3′ position available for covalent binding of a subsequent monomer.

As stated above, in certain embodiments, the fabrication methods may include the depositing of a fluid activator composition which may be deposited on the surface of the support via its own dedicated pulse jet apparatus. A “fluid activator composition” refers to a liquid that includes an amount of activator reagent, where the concentration of activator reagent in the fluid is sufficient to provide for the desired residue, e.g., phosphoramidite, activation during the phosphoramidite synthesis protocol.

The particular activator reagent to be used depends on the particular biopolymer being fabricated. For example, in phosphodiester, phosphotriester and H-phosphonate chemistry, Lewis Acid activators such as sulfonyl halides, sulfonyl azoles, pivaloyl halides, pivaloyl azoles, and adamatane carbonyl halides, are used to form mixed anhydrides that react to for the new internucleotide bond. In the case of phosphoramidite chemistry a protic acid catalyst is used to enhance the rate of displacement of the phosphorus-nitrogen bond. This rate can be additionally enhanced by using an azole catalyst that contains an acidic proton. Protic acid azole activators can include compounds such as, but not limited to, tetrazole, S-ethyl-thiotetrazole, 4-nitrotriazole, 5-benzylthio-tetrazole or dicyanoimidazole, although other acidic azoles can be used. Accordingly, in the case of phosphoramidites, suitable activators include, but are not limited to: tetrazole, S-ethyl tetrazole, dicyanoimidazole (“DCI”), or benzimidazolium triflate. Likewise, a suitable activator in the case of amino acids for polypeptide synthesis is dicyclohexylcarbodiimide (DCC).

The activator solution functions to catalyze the formation of an internucleotide bond, for instance, by the formation of a highly reactive intermediate. Accordingly, the activator is deposited in such a manner to cover feature locations and thereby facilitated the coupling reaction between the solid support and other reactants. An activator compound may be present in a concentration of about 0.05 molar up to about 1.0 molar. The concentration of these activators depends, at least in part, on the solubility of the azole in a solvent that supports phosphoramidite coupling. In certain embodiments, the compositions may include both the precursor and the activator.

Fabrication of a Biopolymer Array

In accordance with the above methods, a chemical array of biopolymers may be fabricated. As described above, in array fabrication, different compositions of the reagents (for instance, the nucleoside monomers and the activator or the preformed nucleic acid or polypeptide) may be deposited at different features (e.g., addresses) on the surface of the solid support during any one cycle so that the different features of the completed array will have biopolymers (e.g., polynucleotides, polypeptides or the like) with different desired biopolymer sequences.

In embodiments of the invention, at least two distinct polymers are produced on different feature regions of the surface of the solid support. By “distinct” is meant that the two polymers differ from each other in terms of sequence of monomeric units. The number of different polymers that are produced on the surface of the solid support may vary depending on the desired nature of the array to be produced, e.g., the desired density of polymeric structures. The product array, therefore, may contain any number of features. For instance, the solid support surface can include a plurality of feature locations, i.e., 2 or more, such as about 10 or more, including about 50 or more, etc., and in certain embodiments, the surface includes 100 or more, 1000 or more, 5000 or more, 10,000 or more, 25,000 or more feature locations.

All of the features may be different, or some or all could be the same. Each feature carries a predetermined moiety or a predetermined mixture of moieties, such as a particular polynucleotide or polypeptide sequence or a predetermined mixture of polynucleotides or polypeptides. The features of the array can be arranged in any desired pattern (e.g. organized rows and columns of features, for example, a grid of features across the substrate surface); a series of curvilinear rows across the substrate surface (for example, a series of concentric circles or semi-circles of features, and the like).

In certain embodiments, the invention provides for the fabrication of arrays with large numbers of very small, closely spaced features. Accordingly, arrays can be fabricated with features that can have widths (that is, diameter, for a round spot) in the range from a minimum of about 10 micrometers to a maximum of about 1.0 cm. In embodiments where very small spot or feature sizes are desired, material can be deposited according to the invention in small spots whose width is in the range about 1.0 micrometer to 1.0 mm, for instance, about 5.0 micrometers to 0.5 mm, such as about 10 micrometers to 200 micrometers. Further, the density of features on the solid support can range from at least about ten features per square centimeter, for instance, at least about 35 features per square centimeter, such as at least about 100 features per square centimeter, at least about 400 features per square centimeter, at least about 1000 features per square centimeter and up to about 10,000 features per square centimeter, or up to 100,000 features per square centimeter. In one embodiment, about 10 to 100 of such arrays can be fabricated on a single solid support (such as glass). In such embodiments, after the solid support has the biopolymer array(s) on its surface, the support can be cut into segments (e.g., of substrates), each of which can carry one or two or more arrays. Interfeature areas which do not carry any polynucleotide may also be present. The interfeature areas could be of various sizes and configurations. It will also be appreciated that there need not be any space separating arrays from one another.

As described above, an aspect of the invention is the application of a dry gas that contacts the surface of a solid support at a feature location before a reagent composition is contacted therewith. The contacting of the feature location (e.g., the surface of the solid support and/or a prior deposited reagent) is in such a manner as to dry the feature location and thereby substantially reduce any moisture that may be present at the feature location. Because moisture is rapidly removed from the feature location by the dry gas, degradation of the synthesis may be avoided.

Accordingly, in certain embodiments, the synthesis reaction takes place without the employment of a coupling chamber. That is, in many synthesis protocols, the above described coupling reaction takes place in a coupling chamber that is used to produce dry or an anhydrous environment. An anhydrous environment may be a large sealed volume through which a dry gas is purged. Instead of requiring an anhydrous environment and employing a coupling chamber and its associated hardware, such as feed lines, regulators and flow conditioners, the methods of the present invention employ a pulse jet head assembly with one or more locally positioned dry gas jets to dry the feature location at which a reagent is to be deposited. Hence, the drying mechanism moves with the pulse jet head assembly and the methods of the invention may be practiced under ambient conditions and without the use of a coupling chamber and/or is associated equipment. However, although the methods of the invention may be practiced without a coupling chamber, they may in fact be employed in conjunction with a suitable coupling chamber.

### Array Fabrication Devices and Systems

As summarized above, the subject invention provides a device for fabricating an array of biopolymers on a surface of a solid support. The subject device includes a fluid dispensing head assembly, for instance, a print jet head assembly, which includes: a) at least one gas jet assembly (e.g., a gas jet) and b) at least one pulse jet assembly (e.g., a pulse jet).

A number of different array fabrication fluid dispensing devices may be modified in accordance with the teachings of the invention for use in the fabrication of a chemical array. One such array fabrication device is a pulse jet fluid deposition device. Pulse jet fluid deposition devices include those described in U.S. Pat. Nos. 4,877,745; 5,338,688; 5,449,754; 5,474,796; 5,658,802; 5,700,637; 5,958,342; 6,242,266; 6,284,465 and 6,306,599, herein incorporated by reference in their entirety.

Accordingly, in certain embodiments, the fluid dispensing head assembly of the invention is a pulse jet head assembly. An aspect of the pulse jet fluid dispensing devices of the present invention is that the pulse jet head assembly has been modified to include a gas jet assembly in addition to a pulse jet assembly.

A gas jet assembly may include one or more of: a gas source, a gas delivery element (e.g., a gas jet), a gas delivery conduit, and/or a gas flow control element (for instance, a valve). Any suitable gas source may be used. For instance, gas source may be a container that includes a reservoir of gas or may be a gas line connected to a suitable reservoir of gas. The gas may be any non-reactive gas suitable for contacting the surface of a solid support and producing a local dry area thereon. In certain embodiments, the gas may be a reactive gas. The gas may be an anhydrous (e.g., dry) gas under conditions of use. In certain embodiments, the dry gas has a water content that is less than about 0.2 ppm by volume, for instance less than about 0.1 ppm by volume, including less than about 0.05 ppm by volume. Examples of dry gas include: one or more of nitrogen, helium, a noble gas, such as: argon, krypton, xenon and neon. The gas within the reservoir may be under pressure. If the gas is not under pressure, a suitable pump may be included for pumping the gas from the gas source to a gas delivery element.

The gas jet assembly may include a gas delivery element, for instance, a gas jet. The gas jet may include a reservoir, a valve and/or an orifice. The diameter of the orifice may be from about 0.01 μm to about 1 mm, for instance, about 0.1 μm to about 500 μm, such as about 1 μm to about 100 μm, and including 10 μm to about 50 μm. The orifice may be a single opening or a multiplicity of openings. The design of the gas jet assembly orients the gas delivery element opposite a solid support such that the gas to be delivered is directed substantially vertically onto the solid support and thereby dries the contacted area. In certain embodiments, the gas jet assembly is positioned to direct a gas substantially normal to the solid support. That is the gas is directed vertically in a direction such that the gas contacts the surface of the solid support in a manner sufficient to rapidly dry a localized portion of the solid support. By “localized portion” is meant a discrete portion of the solid support onto which a fluid reagent is to be deposited.

The gas may be delivered under pressure and in such a manner so as to provide a careful distribution and controlled unidirectional flow. The pressure differential behind the gas delivery element forces a defined stream or jet of gas out of the pulse jet head assembly. Accordingly, the flow of gas may fall within the following parameters: a velocity that ranges from about 0.01 cm/s to about 1000 cm/s; a pressure that ranges from about 1 psi to about 0.15 psi; a density that ranges from about 0.0005 gm/cm to about 0.0015 gm/cm; and a temperature that ranges from about 0° C. to about 100° C.

The gas jet assembly may also include a gas delivery conduit. The gas delivery conduit may be any element suitable for delivering a gas from the gas source to the gas delivery element. For instance, in certain embodiments, the gas delivery conduit is a gas line having one or more of an inlet and an outlet portion, an outlet opening, and a connection element that communicates with the gas delivery element. The gas delivery conduit functions to direct the flow of a gas, e.g., an anhydrous gas, such as nitrogen, from the gas source, through the gas delivery conduit and to the gas delivery element. The flow of the gas can be regulated by a suitable valve, e.g., a venturi valve and/or a flow regulator.

Accordingly, a gas jet assembly may further include one or more gas flow control elements for actuating one or more gas jets. Accordingly, a gas flow control element may be any element suitable for actuating one or more gas jets of the device and/or finely controlling one or more parameters of the flow of the gas to be delivered to the surface of the solid support. Hence, by operation of the one or more gas flow control elements, the flow, amount, time and dimensions of a gas to be delivered to the surface of the substrate can be controlled. The flow of gas that exits from the gas delivery element may be a steady stream or in one or more burst. Accordingly, the gas may be delivered over a time range from about 0.1 to about 5 s.

In certain embodiments, the gas flow control element may include one or more of a valve, a compressor and/or a flow regulator. For instance, one or more valves may be used for controlling the flow of gas from the gas source to the gas delivery element. A valve may be any type of valve suitable for controlling the level and flow of gas through and out of the gas jet assembly. A suitable valve may be, for example, a venturi valve. Other valves that may be employed include electrically operated directional valves or proportional valves and so forth. Hence, with the use of valves, the flow rate and pressure of the gas may be controlled. Accordingly, the flow and pressure of gas out of the gas delivery element can be finely tuned to desired parameters.

As will be described in greater detail herein below, one or more valves of the system may be controlled by a controller which adjusts the flow and pressure of the gas. Such controllers may include, for example, a venturi control valve or an electronic controller programmed to take feedback from a sensor and open and close or modulate the valve, thus, controlling the pressure. The controller can include software running on any convenient hardware, e.g., a PC or MAC. A sensor may also be included. Suitable sensors for use with the valves and controllers of the invention include humidity sensors, pressure sensors, flow sensors, low pressure manometers, pressure transducers, and the like.

In certain embodiments, the gas flow control element may include a compressor. A compressor element functions to compress the flow of a gas and thereby increases the pressure of the gas flow. The compressor may be a mechanical/electrical element or a structural element that acts to further compress and/or direct the flow of the gas. For instance, the compressor may be a MEMS element or a structural element such as one or more passageways of decreasing diameter through which the gas flows and is thereby compressed (e.g., passageways of decreasing diameter, which may be of nanometer dimensions).

As set forth above, an aspect of the pulse jet fluid dispensing devices of the present invention is a pulse jet head assembly that includes a pulse jet assembly. A pulse jet assembly may include one or more of: a reservoir, a dispensing chamber, a dispensing orifice, an ejector, a fill port and/or other additional elements that function to facilitate the delivery of a reagent fluid to a dried feature location on the surface of a solid support.

Accordingly, a pulse jet assembly of the present invention may include one or more reservoirs for holding a fluid. A reservoir may include one chamber for holding a single fluid, or multiple chambers for holding a multiplicity of fluids. For instance, the reservoir may contain a fluid reagent such as an activator reagent or a biopolymer monomeric precursor reagent (e.g., such as a nucleoside or amino-acid reagent).

Additionally, a pulse jet assembly of the present invention may include one or more dispensing chambers for dispensing a fluid reagent. The reservoir may be in fluid communication with the dispensing chamber. Hence, the dispensing chamber may be part of the reservoir or may be physically separated from the reservoir, but in such a case a means for ensuring fluid communication between the two is provided. Accordingly, the number of dispensing chambers may be equal to the number of reservoirs, or alternatively the number of dispensing chambers may be more than the number of reservoirs, for instance, wherein a plurality of dispensing chambers service a single reservoir. That is a single reservoir may have multiple delivery chambers, orifices and ejectors. For example, the number of delivery chambers, orifices and ejectors may be from about 1 to about 1,000, for instance about 10 to about 500, including about 50 to about 200, depending on the size and the materials used to construct the pulse jet head.

Further, a pulse jet assembly of the present invention may include one or more dispensing orifices. The dispensing orifice may in fluid communication with a dispensing chamber through which a fluid is dispensed. The orifice may have any suitable configuration, for instance, the orifice may taper inwardly away from the dispensing chamber and toward the open end of the orifice. The orifice may be of any suitable size but should be such that it produces a spot of suitable dimensions on the surface of the solid support. For instance, an orifice of the invention may have an exit diameter (or exit diagonal depending upon the particular format of the device) in the range about 1 μm to 1 mm, usually about 5 μm to 100 μm, and more usually about 10 μm to 60 μm. The reservoir chamber and the connected dispensing chamber, with which the orifice communicates, together may have a combined fluid capacity in the range of about 1 μL up to about 1 mL, for instance, less than 100 μL, including about 0.5 μL to about 10 μL, such as about 1 μL to about 5 μL.

A pulse-jet assembly of the present invention may also include a suitable ejector. For instance, an ejector operatively associated with a dispensing orifice may be included. The ejector may be electrically connected to an electrical energy source that can be controlled to deliver a suitable pulse of electrical energy to activate the ejector on demand so as to eject at least one drop of a fluid out of the orifice. Any suitable element capable of converting an electrical charge to a fluid impulse may be used. For instance, a suitable ejector may be an electrical resistor, which operates as a heating element. In certain embodiments, a suitable ejector may be a heating element, a piezoelectric ejector, or the like.

Where the ejector may include a heating element, the heating element may be made out of a material that can deliver a quick energy pulse, such as TaAl and the like. The heating element is capable of achieving temperatures sufficient to vaporize a sufficient volume of fluid in the dispensing chamber so as to produce a bubble of suitable dimensions upon actuation of the ejector. For instance, the heating element is capable of attaining temperatures at least about 100° C., for instance, at least about 400° C., including at least about 700° C., and may be as high as 1000° C. or higher.

The pulse jet assembly may also include other elements, for instance, a fill port, a filter and a gas source and valve. Specifically, a fill port in fluid communication with the reservoir may also be included. In use, a sample fluid, such as a reagent, may be loaded into the reservoir through the fill port. Alternatively, depending on the design of the pulse jet head assembly and the properties of the fluid to be dispensed, fluid can also be loaded into the reservoir through the dispensing orifice. The dispensing orifice sizes may be in the order of tens of microns while fill ports can be as large as or larger than thousands of microns.

One or more filters may optionally be provided. For instance, the dispensing orifice can act as a filter or one or more filters may be included in the pulse jet assembly for filtering the exiting fluid by retaining particulates, agglomerates, impurities or other solids in the pulse jet.

A gas source, conduit and valve can also be used to provide a slightly negative spotting pressure so as to retain the reagent fluid within the pulse jet in the absence of the activation of the ejector. For instance, a gas source connected to a gas line having an inlet and an outlet portion, an outlet opening, and a throat element that communicates with the reservoir may also be provided. In this manner a flow of a gas, e.g., an anhydrous gas, such as nitrogen, may be directed from the gas source, through the gas delivery conduit and to the gas to the reservoir so as to provide a sufficient back pressure to retain the reagent fluid within the pulse jet in the absence of the activation of the ejector. The flow of the gas can be regulated by a suitable valve, e.g., a venturi valve and/or a flow regulator.

The pulse jet head assembly of the invention may further include appropriate electrical and mechanical architecture and electrical connections, wiring and devices such as timers, clocks, and so forth for operating the various elements of the apparatus.

When the print head assembly is in operation, a fluid reagent from a reservoir flows into the dispensing chamber where energy is applied to the fluid reagent. The energy can be applied in a variety of ways, such as through piezoelectric or thermal means. As a result, an amount of the fluid reagent is ejected from the dispensing chamber through the dispensing orifice. The amount of fluid reagent may vary, for instance, each fluid reagent droplet may have a volume of from about 0.1 to about 1000 μL.

The design of the pulsejet head assembly is such that it orients the dispensing orifice opposite a solid support such that the fluid reagent to be dispensed is ejected onto the substrate. The dimensions and configuration of the pulse jet is such that it is capable of producing and depositing a droplet onto the surface of the solid support at a defined feature.

As summarized above, an aspect of the invention is a pulse jet head assembly that includes both a gas jet and pulse jet. Accordingly, the pulse jet head assembly may include a single gas jet assembly (e.g., a single gas jet) and a single pulse jet assembly (e.g., a single pulse jet). Alternatively the pulse jet head assembly may include a plurality of gas jet assemblies and/or a plurality of pulse jet assemblies. As such, the assembly may include a single gas jet and a plurality of pulse jets, a plurality of gas jets and a single pulse jet, or a plurality of gas jets and a plurality of pulse jets (where plurality means 2 or more).

In certain embodiments, the pulse jet head assembly includes at least one gas jet that is positioned adjacent to a pulse jet. In certain embodiments, the pulse jet head assembly includes at least two gas jets and at least one pulse jet. For instance, the pulse jet head assembly may include two gas jets that are separated one from the other by an intervening pulse jet. That is the gas jets are adjacent to opposite sides of the pulse jet.

In certain embodiments, the pulse jet head assembly comprises a plurality of gas jets and a plurality of pulse jets wherein the gas jets and pulse jets are aligned and positioned in one or more rows or stacks. Accordingly, in one embodiment, at least one gas jet is positioned at the beginning and one gas jet is positioned at the end of a row of jets. In another embodiment, the plurality of gas jets are separated from one another by an intervening gas jet.

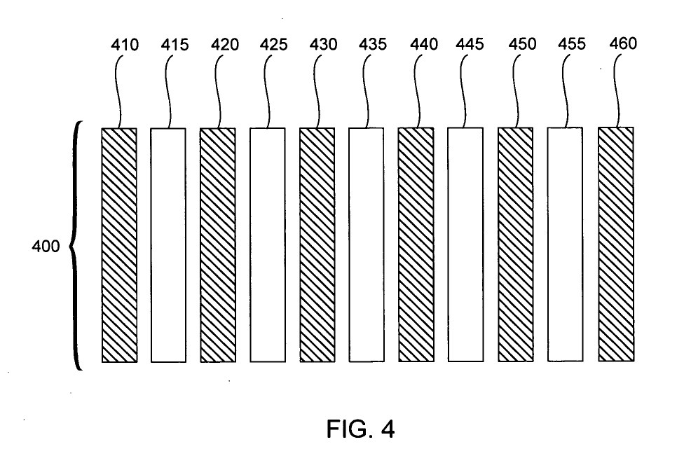

For instance, as can be seen with reference to FIG. 4, a row of jets 400 may include 6 or more gas jets (elements 410, 420, 430, 440, 450 and 460) and 5 or more pulse jets (elements 415, 425, 435, 445 and 455), wherein the row begins with gas jet 410, ends with gas jet 460, and has intervening pulse jets 420, 430, 440 and 450 that separate pulse jets 415, 425, 435 and 455 one from another.

Although the above has been described with reference to a single row or stack of jets with a specific number of gas and pulse jets in a particular configuration (e.g., a single line or row), it is understood that the number and configuration of jets may vary. For instance, a pulse jet head assembly could include 7 gas jets with 6 intervening pulse jets, or the gas and/or pulse jets could be configured as a plurality of rows (for instance, two, three, four, five, six or more rows), a square, circle, ellipse, triangle, or any other suitable configuration based on the demands and configuration of the system.

Accordingly, in certain embodiments, the pulse jet head assembly may include a plurality of gas jets that are arranged in a single row and/or may include a plurality of pulse jets arranged in a single row, for instance, a row of pulse jets that is adjacent to a row of gas jets. In certain embodiments, the pulse jet head includes two rows of gas jets separated by a row of intervening pulse jets. In certain embodiments this configuration is repeated multiple times, for instance, one, two, three or more times. Accordingly, in certain embodiments, the pulse jet head assembly may include N rows of pulse jets and N+1 rows of gas jets, for instance, where the rows of pulse jets separate the rows of gas jets one from another.

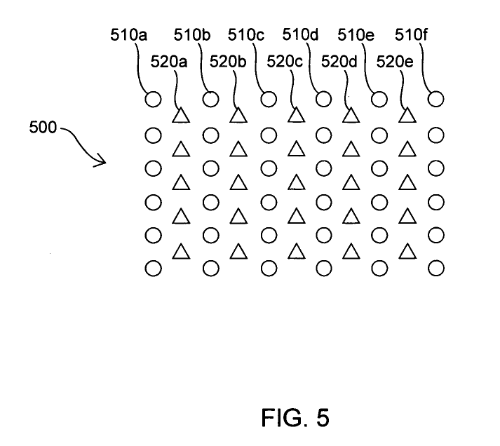

For example, as can be seen with reference to FIG. 5, in one embodiment, the pulse jet head assembly (500) includes five rows of pulse jets (elements 520a-e) and six rows of gas jets (elements 510a-f). As shown in this embodiment, the rows of pulse jets separate the rows of gas jets one from the other. However, the rows of gas jets and pulse jets may be in any suitable configuration.

Although the above has been described with reference to a single pulse jet head assembly containing both gas jet assemblies (gas jets) and pulse jet assemblies (pulse jets) on a single pulse jet head assembly, a device of the invention may include more than one pulse jet head assembly, for instance, a stack of pulse jet head assemblies (e.g., two, three, four, five, six or more) which may each contain both gas jet assemblies (gas jets) and pulse jet assemblies (pulse jets) or the pulse jet head assemblies of the stack may include only one type of jet (e.g., only gas jets or only pulse jets).

### Systems

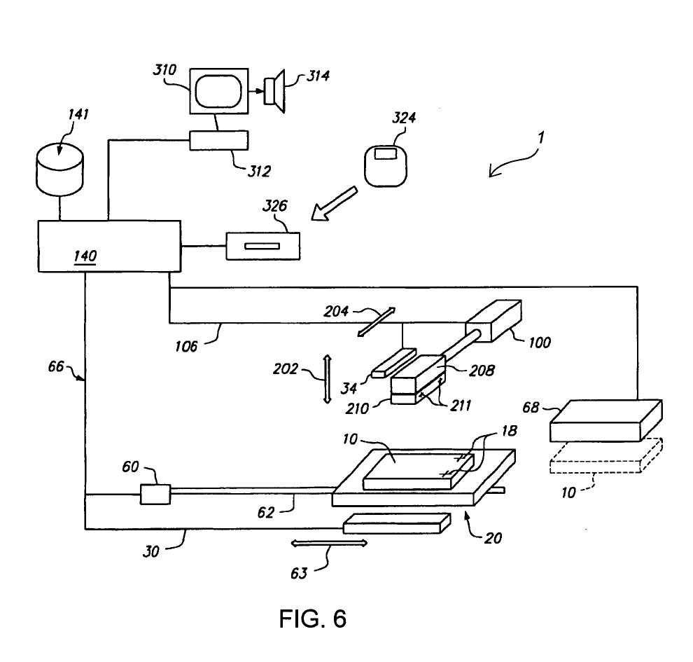

In another aspect of the invention, a system for producing an array of biopolymers on the surface of a solid support is provided. As can be seen with reference to FIG. 6, there is shown a system according to one aspect of the invention that includes a solid support (e.g., substrate) station 20 on which can be mounted a solid support 10. Pins or fiducials 18 or similar means can be provided on the solid support station 20 by which to approximately align support 10 to a position thereon. Support station 20 can include a vacuum chuck connected to a suitable vacuum source (not shown) to retain a support 10 without exerting too much pressure thereon, since support 10 may be made of glass. In one aspect, a flood station (flow cell) 68 is provided which can expose the entire surface of the support 10, when positioned in the station 68, as illustrated in broken lines in FIG. 6, to a fluid used in the fabrication process, and to which all features must be exposed during a fabrication cycle (for example, in the case of in situ fabrication: oxidizer, deprotection agent, and/or wash buffer).

In one aspect, a pulse jet head assembly 210 of stacked gas jet and pulse jet assemblies is retained by a head retainer 208. Head assembly 210 may also contain-fiducials 211. In certain aspects, the pulse-jet head assembly includes gas jets that are operatively connected to an anhydrous gas source (not shown) and pulse-jets which are operatively connected to respective reservoirs of fluid reagents (not shown). A positioning system includes a carriage 62 connected to a first transporter 60 controlled by a processor 140, and a second transporter 100 controlled by processor 140. Transporter 60 and carriage 62 are used to execute one axis position of station 20 (and hence mounted support 10) facing the dispensing head assembly 210, by moving it in the direction of arrow 63, while transporter 100 is used to provide adjustment of the position of head retainer 208 (and hence head assembly 210) in a direction of axis 204.

In this manner, head assembly 210 can be moved line-by-line, by moving the head assembly 210 along a line over a support 10 in the direction of axis 204 using transporter 100, while line by line movement of support 10 in the direction of axis 63 is provided by transporter 60. Transporter 60 can also move support holder 20 to position the support 10 beneath flood station 68. Head assembly 210 may also be moved in a vertical direction 202, by another suitable transporter (not shown). It will be appreciated that other configurations could be used. It will also be appreciated that both transporters 60 and 100, or either one of them, with suitable construction, could be used to perform the foregoing motion of the head assembly 210 with respect to the support.

Thus, when the present invention recites “positioning” one element (such as head assembly 210) in relation to another element (such as support 10 or one or more stations 68) it will be understood that any required moving can be accomplished by moving either element or a combination of both of them. The head assembly 210, the positioning systems, and processor 140, together act as the deposition system of the device 1 in accordance with the present invention. An encoder 30 provides data on the exact location of the holder 20 and also the head assembly position. Any suitable encoder, such as an optical encoder, may be used which provides data on linear position.

As described above, each gas jet assembly of the head assembly may be associated with a single gas source through individual gas delivery elements or may be associated with individual gas sources. Each gas jet assembly is also associated with a corresponding set of one or more gas orifices that are operatively connected to the gas delivery element via a suitable gas delivery conduit and connectors. One or more suitable gas flow control elements, such as a venturi valve may also be included so as to control the pressure and other flow dimensions of the gas from the gas source, through the delivery element and out of the orifice.

Each pulse jet assembly of the head assembly is associated with a corresponding reagent reservoir and a set of one or more drop-dispensing orifices and ejectors, which are positioned in the chambers opposite the respective orifices. The pulse jet assemblies may be any convenient pulse jet including, such as a thermal pulse jet assembly, a piezoelectric pulse jet assembly, etc. While the following additional description is provided primarily in terms of a thermal pulse jet head device, piezoelectric devices may be used in certain embodiments and come within the scope of the invention.

In one aspect, the one or more valves of each gas jet element are under the control of a processor 140. Each gas jet orifice, which may be associated with a reservoir and/or one or more additional gas flow control elements (e.g., a valve or the like), defines a corresponding gas jet assembly. Additionally, each pulse jet ejector is in the form of an electrical resistor operating as a heating element under control of a processor 140. It is noted that piezoelectric elements are also of interest an may be employed. Each pulse jet orifice with its associated ejector and portion of the chamber, defines a corresponding pulse jet assembly.

Processor 140 functions to control the delivery of an anhydrous gas from each gas delivery orifice of the gas jet in the pulse jet head assembly. Additionally, Processor 140 controls the application of electrical pulses to the ejectors of the pulse jet assemblies. The application of a single electric pulse to an ejector will cause a droplet to be dispensed from a corresponding orifice. Certain elements of the gas and pulse jet assemblies of the head assembly 210 can be adapted from parts of a commercially available thermal inkjet head device, such as available from Hewlett-Packard Co. as part no. HP51645A. Alternatively, multiple heads could be used instead of a single head and being provided with respective transporters under control of processor 140 for independent movement. In this alternative configuration, each head assembly may dispense a gas and a corresponding monomer or activator or separate head assemblies can be used to dispense the gas and the fluid reagent.

The amount of fluid that is expelled in a single actuation event of a jet assembly can be controlled by changing one or more of a number of parameters, including the orifice diameter, the orifice length (thickness of the orifice member at the orifice), the size of a reservoir chamber, and the size of the heating element (e.g., in a pulse jet), along with others. For instance, the amount of a reagent fluid that is expelled from a pulse jet assembly during a single actuation event may be in the range of about 0.1 μL to 1000 μL, that is about 0.5 to about 500 μL, including about 1.0 to about 250 μL. The velocity at which a fluid reagent is expelled from the pulse jet may be more than about 1 m/s, more than about 10 m/s, and may be as great as about 20 m/s or greater. As will be appreciated, if the orifice is in motion with respect to the receiving surface at the time an ejector is activated, the actual site of deposition of the material will not be the location that is at the moment of activation in a line-of-sight relation to the orifice, but will be a location that is predictable for the given distances and velocities.

As mentioned above, the pulse jet head assembly of the system depicted in FIG. 6 may include single row or a plurality of rows of gas jets and reagent-dispensing jets that are operatively connected to respective sources of fluid compositions. Both the gas jet and the pulse jet assemblies can be activated to deliver a volume of fluid (e.g., a volume of gas or a reagent monomer, etc.) to each feature location as desired and/or according to programmed instructions, e.g., such as instructions that may be included on a disc 324. Once the pulse-jet head assembly has traveled across the desired length or width of the solid support (e.g., a substrate), the pulse-jet head assembly can be stepped to start a new row(s).

In certain embodiments, the number of pulse jets in a given head includes that required for delivery of monomers as well as an additional pulse jet assembly for the delivery of an activator reagent. In certain embodiments, the number of gas jets is determined by the formula (n+1), wherein n equals the number of pulse jets. In certain embodiments, each pulse jet is separated by an intervening gas jet. For instance, where the number of pulse jets is five (e.g., one for each nucleoside monomer reagent and one for an activator reagent) the number of gas jets will be six. The jets may be in any suitable configuration, but in one embodiment are configured as a row of jets where in the row begins and ends with a gas jet. See, for instance, FIG. 4. In other embodiments, the jets are configured in a plurality of rows, as seen with reference to FIG. 5.

The device may further include a display 310 and an operator input device 312. The operator input device may be, for example, a keyboard, mouse, or similar input devices. Processor 140 has access to a memory 326, and controls the pulse-jet head assembly 210, (specifically the activation of the valves and ejectors therein), operation of the positioning system, operation of each jet assembly disposed in the pulse jet head assembly, and operation of the display 310. Memory 326 may be any suitable device in which processor 140 can store and retrieve data 324, such as magnetic, optical, or solid-state storage devices. Processor 140 may include a general purpose digital microprocessor suitably programmed from a computer readable medium carrying necessary programming code, to execute all of the steps required by the present invention, or any hardware or software combination which will perform the equivalent steps. The programming may be provided remotely to processor 140, or previously saved in a computer program product such as memory 326 or some other portable or fixed computer readable medium using any of those devices mentioned above.

Programming for controlling the device according to the present invention is also provided. For example, the programming may control the device to flood the substrate surface with activator fluid, and then remove the fluid from the surface in a manner that results in the production of solid activator in feature locations of the surface, as reviewed above. The programming may further control the pulse jet head and jet assemblies to form an array according to an input selection from a user, e.g., the user may be presented through a graphical interface multiple choices of types of arrays that may be formed. The user, through an input device, may choose the type of array to be formed, wherein the programming controls the device according to the present invention to form the array selected by the user.

Operation of the device will now be described in accordance with aspects of the methods described herein above. In one aspect, memory 326 holds instructions for providing a target drive pattern for a target array and which can include, for example, target locations and dimensions for each spot or feature on support 10. The instructions can further include, for example, movement commands which can be executed by transporters 60 and 100 as well as firing commands for each of the gas jets and pulse jets disposed in head assembly 210 coordinated with the movement of head assembly 210 and support 10. In one aspect, this target drive pattern is based upon a desired target array pattern and can have either been input from an appropriate source (such as input device 312, a portable magnetic or optical medium, or from a remote server, any of which may communicate with processor 140), or may have been determined by processor 140 based on an input target array pattern (using any of the appropriate sources previously mentioned) and the previously known operating parameters of the apparatus. The memory holding instructions for providing a target drive pattern can further include instructions to head assembly 210 and positioning system of the device to deposit a dry gas, activator and biopolymer reagent at each region at which a feature is to be formed.

BRET: We should probably increase the number of printheads anticipated in a set to more than five and perhaps up to 25 say to include protein synthesis and analogue (sp) bases and cleavable linkers.

Where the number of pulse jets in a row is five, four of the pulse jets can be loaded with four different monomer reagents and one pulse jet can be loaded with an activator reagent. Of course, only four pulse jets (one for each monomer) may be included as part of the pulse jet head assembly. Alternatively, more than 5 pulse jets may be present, e.g., 10 or more, 15 or more, 20 or more, 25 or more, 30 or more etc. For example, where the polymers to be synthesized are proteins, one may include 25 or more jets to accommodate the larger number of monomeric reagents. In this instance, an activator solution can be contacted with the surface of the solid support by a different means, for instance, via a suitably configured flood station (e.g., element 68). In one aspect, the flood station 68 is loaded with all necessary solutions, which may include an oxidizer, deprotection agent, wash buffer and/or fluid activator composition (if not included in a separate pulse jet assembly). Operation of the following sequences are controlled by processor 140, following initial operator activation, unless a contrary indication appears.

In one embodiment, for any given support 10, a support 10 is loaded onto the solid support station 20, either manually or automatically. A target drive pattern necessary to obtain a target array pattern, is determined by processor 140 (if not already provided), based on operating parameters of the device. In one aspect, the device is then operated as follows: (a) if not the first cycle, position support 10 at flood station 68 and for all regions of the arrays being formed, deprotect previously deposited and linked monomer on support 10; (b) move support 10 to receive droplets from head assembly 210; (c) activate gas jet to direct a volume of gas substantially vertically so as to contact feature location(s) with the volume of gas and thereby substantially dry the contacted feature locations; (d) optionally activate pulse jet to dispense a volume of reagent activator composition onto feature location (if included); (e) optionally activate gas jet to direct a volume of gas vertically so as to contact feature location(s) containing activator; (f) activate pulse jet to deposit droplet(s) so as to dispense a volume of appropriate next monomer onto feature location, such that the first linking group is activated by the activator and links to previously deprotected monomer; (g) move substrate 10 back to flood station 68 for oxidation, capping and washing steps over entire substrate; (h) activate gas jet to direct a volume of gas so as to contact feature location(s) containing monomer and (g) repeat foregoing cycle for all of the regions of all desired arrays until the desired arrays are completed. It is to be noted, that if an activator reagent pulse jet is not included as part of the pulse jet head assembly, then steps (d) may be omitted and an activator reagent may be contacted with the surface of the solid support as needed at flood station 68, using the appropriate protocol.

### Utility

The above protocols of the invention produce chemical, e.g., biopolymer ligand, arrays that can be employed in a variety of different applications. Whether the biopolymers (e.g., ligands) are deposited onto the surface of the solid support in premade form or produced on the surface in situ by deposition of precursors thereof, as described above, a common step to both approaches is the production of two or more ligands on the functionalized surface of a solid support.

Chemical arrays produced as described above find use in a variety of different applications, where such applications include analyte detection applications in which the presence of a particular analyte in a given sample is detected at least qualitatively, if not quantitatively, array CGH assays, location analysis assays, nucleic acid synthesis applications, genotyping assays, and the like.

For instance, for analyte detection applications, the sample suspected of comprising the analyte of interest is contacted with an array produced according to the subject methods, using the subject devices, under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g. through use of a signal production system, e.g. an isotopic or fluorescent label present on the analyte, etc. The presence of the analyte in the sample is then deduced from the detection of binding complexes on the surface of the solid support.

Specific analyte detection applications of interest include hybridization assays in which the nucleic acid arrays of the subject invention are employed. In these assays, a sample of target nucleic acids is first prepared, where preparation may include labeling of the target nucleic acids with a label, e.g. a member of signal producing system. Following sample preparation, the sample is contacted with the array under hybridization conditions, whereby complexes are formed between target nucleic acids that are complementary to probe sequences attached to the array surface. The presence of hybridized complexes is then detected. Specific hybridization assays of interest which may be practiced using the subject arrays include: gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992. Also of interest are U.S. Pat. Nos.: 6,656,740; 6,613,893; 6,599,693; 6,589,739; 6,587,579; 6,420,180; 6,387,636; 6,309,875; 6,232,072; 6,221,653; and 6,180,351. In certain embodiments, the subject methods include a step of transmitting data from at least one of the detecting and deriving steps, as described above, to a remote location.

Where the arrays are arrays of polypeptide binding agents, e.g., protein arrays, specific applications of interest include analyte detection/proteomics applications, including those described in U.S. Pat. Nos.: 4,591,570; 5,171,695; 5,436,170; 5,486,452; 5,532,128 and 6,197,599 as well as published PCT application Nos. WO 99/39210; WO 00/04832; WO 00/04389; WO 00/04390; WO 00/54046; WO 00/63701; WO 01/14425 and WO 01/40803—the disclosures of which are herein incorporated by reference.

As such, in using an array made by the method of the present invention, the array will be exposed to a sample (for example, a fluorescently labeled analyte, e.g., nucleic acid or protein containing sample) and the array is then read. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER available from Agilent Technologies, Palo Alto, Calif. Other suitable apparatus and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934.

However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample or an organism from which a sample was obtained exhibits a particular condition). The results of the reading (processed or not) may be forwarded (such as by communication) to a remote location if desired, and received there for further use (such as further processing).

In certain embodiments, the methods include a step of transmitting data from at least one of the detecting and deriving steps, as described above, to a remote location. By “remote location” is meant a location other than the location at which the array is present and hybridization occur. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as signals (e.g., electrical, optical, radio signals, and the like) over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

As such, in using an array made by the method of the present invention, the array will be exposed to a sample (for example, a fluorescently labeled analyte, e.g., nucleic acid- or protein-containing sample) and the array will then be read. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER scanner available from Agilent Technologies, Palo Alto, Calif. Other suitable apparatus and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934; the disclosures of which are herein incorporated by reference.

However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

### Kits

In accordance with the invention, kits are provided for retrofitting an existing chemical array fabrication apparatus. The subject kits at least include a fluid dispensing head assembly that contains at least one gas jet assembly and one pulse jet assembly. In certain embodiments, the fluid dispensing head assembly is a pulse jet head assembly that includes a plurality gas jets and pulse jets, for instance, a plurality of rows of gas jets and pulse jets, wherein the number of rows of pulse jets is equal to N and the number of rows of gas jets is equal to N+1. In certain embodiments, the rows of pulse jets separate the rows of gas jets. The components of the kit may also include suitable gas or reagent delivery conduits, connectors, reservoirs, storage elements, valves, pumps, subject gas sources containing gasses, reagents and the like. The various components of the kit may be present in separate containers or certain compatible components may be combined into a single container, as desired.

In addition to above-mentioned components, the subject kits may further include instructions for retrofitting an array fabrication apparatus with the pulse jet head assembly and for using the components of the kit to practice the subject methods. The instructions may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc.

In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

The above discussion demonstrates a new pulse jet print head assembly and a method for producing a biopolymer array. The devices and methods disclosed herein are readily adaptable for use with prior art printing devices and the methods of their use can be tailored to produce an array of desired properties. Prior art devices can be modified to contain the disclosed fluid dispensing head assemblies, and their performance will be improved greatly form this modification. Accordingly, the subject system represents a significant contribution to the art.

The following examples are offered by way of illustration and not by way of limitation.

### EXPERIMENTAL

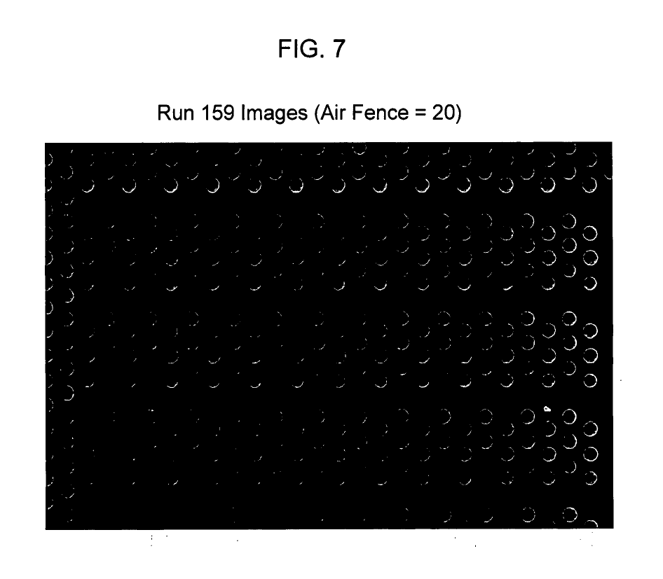

A variety of biopolymer microarrays were fabricated and tested. The microarrays were fabricated using four different protocols. In one protocol, Run 159, the arrays were fabricated on the surface of a solid support by contacting a feature location on the surface of the solid support with a vertically directed dry nitrogen (N₂) gas prior to dispensing a fluid reagent onto the dried feature location. The flow rate of the gas was set to 20 liters/per minute (LPM) and the arrays in this run group were fabricated without the use of an anhydrous chamber. As can be seen with reference to FIG. 7, the post run test prints indicate the jetting was fine, however, some small spots were mildly and randomly out of line, however, a wipe of the print heads corrected this issue.

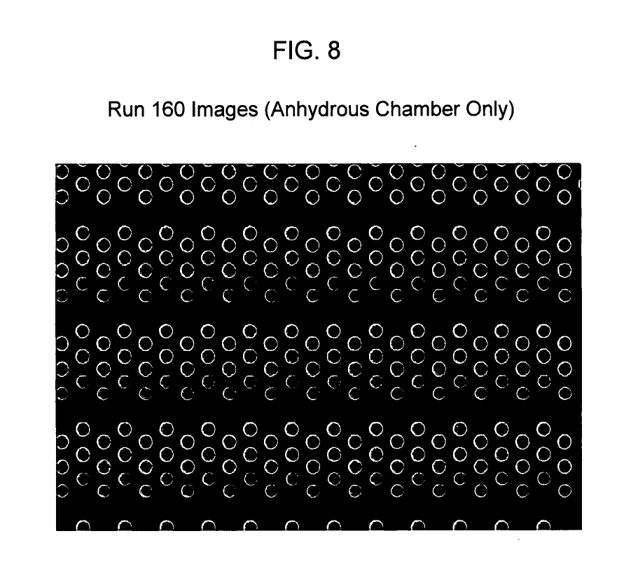

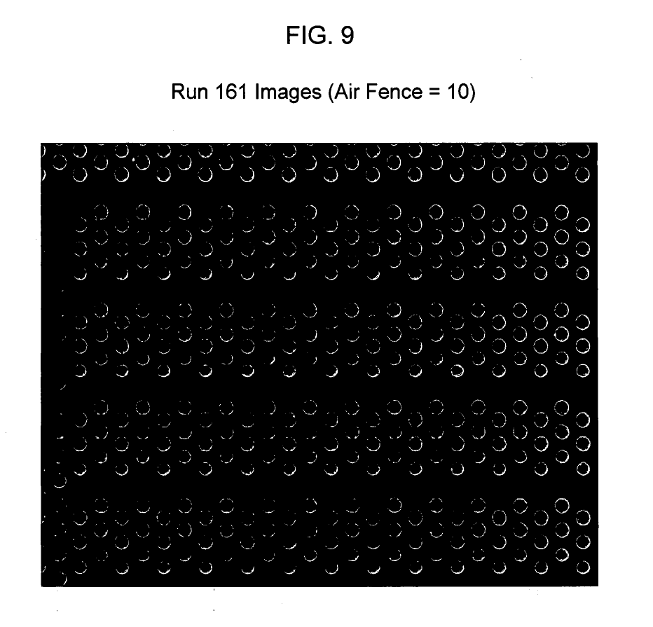

In another protocol, Run 161, the arrays were fabricated in the manner set forth in Run 159, however, the gas flow rate was set at 10 LPM. As can be seen with reference to FIG. 8, the post run test prints indicated that the jetting was fine and the signals were both strong and comparable to the signals obtained from fabricating the arrays within an anhydrous chamber alone. See Run 162 and FIG. 9, below.

In a further protocol, Run 160, the arrays were fabricated on the surface of the solid support in the conventional manner, that is within an anhydrous chamber and without contacting feature locations with a vertically directed dry gas so as to dry the feature locations prior to dispensing a fluid reagent. As can be seen with reference to FIG. 9, the post run test prints indicated that the jetting was fine and the signal was strong.

In a final protocol, Run 163, the arrays were fabricated both within an anhydrous chamber and with contacting the feature locations on the surface of the solid support with a vertically directed dry nitrogen (N₂) gas prior to dispensing a fluid reagent onto the dried feature location. The flow rate was set at 10 (LPM). The post run test indicated that the jetting was fine.

The arrays of all other Runs were fabricated using an anhydrous chamber only.

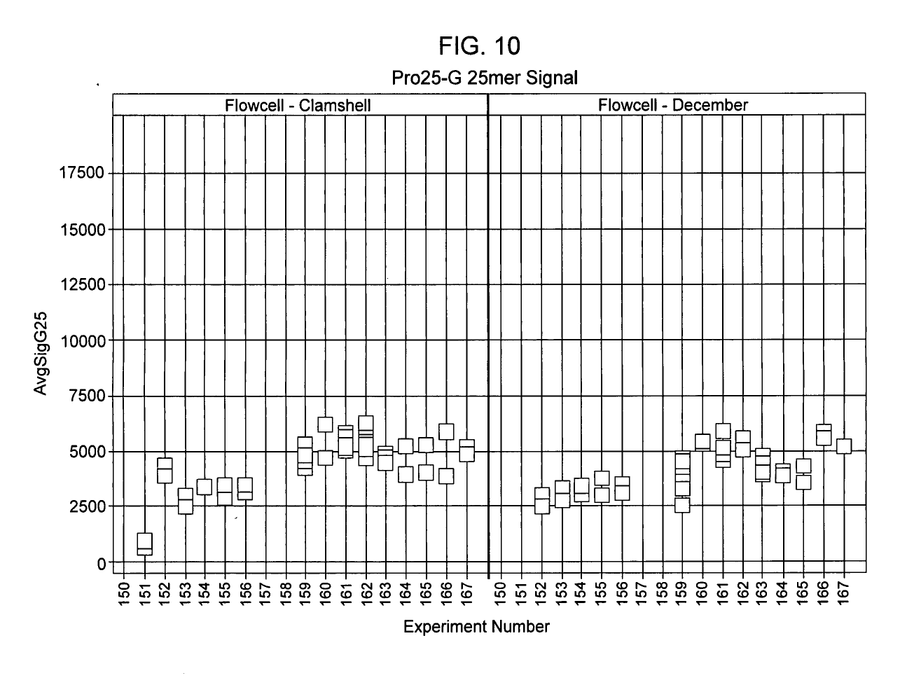

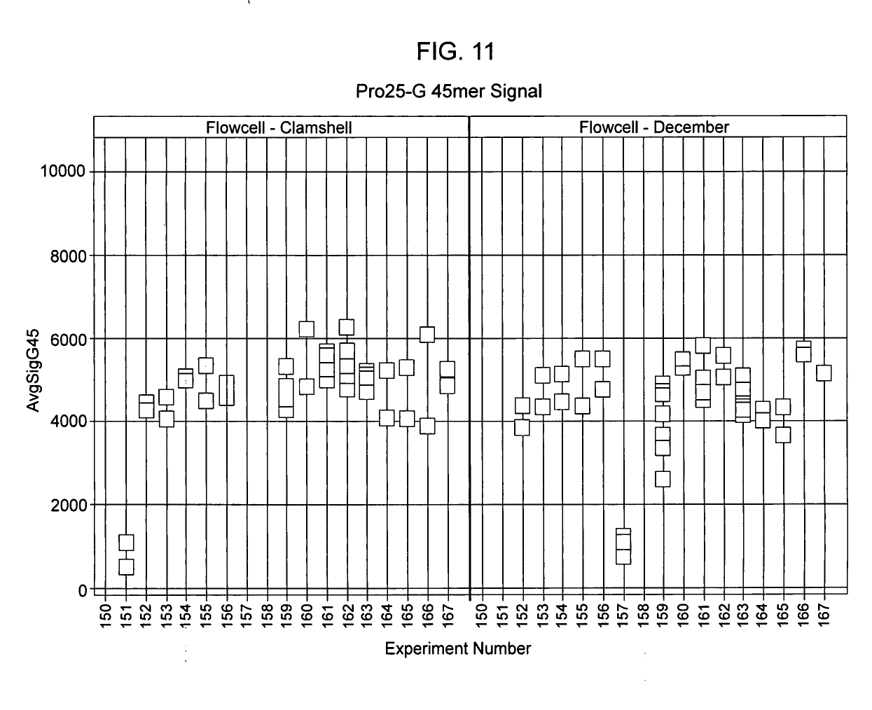

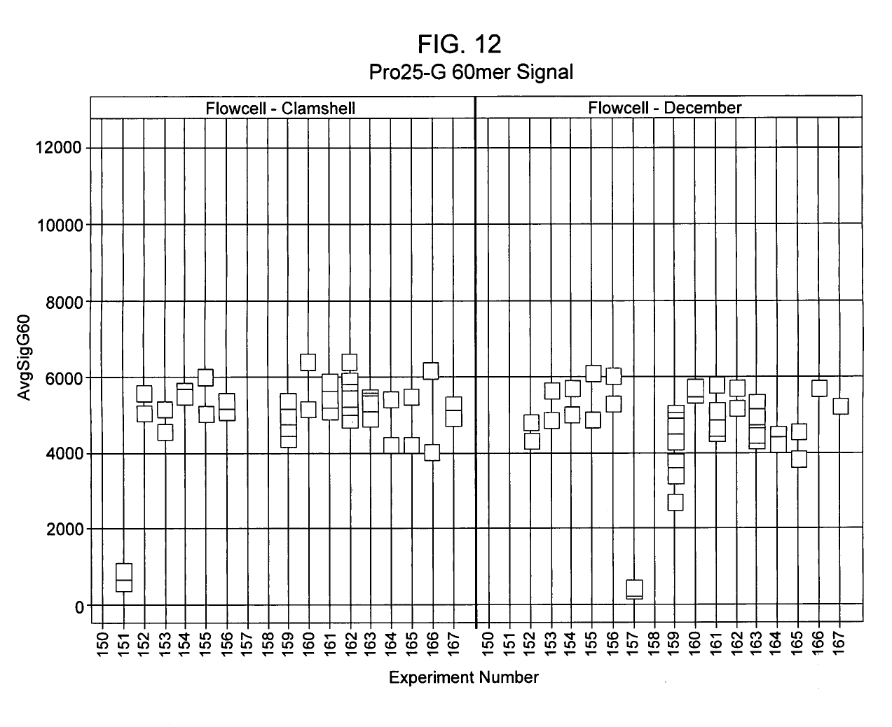

The above protocols were employed to fabricate arrays of biopolymers of different lengths, including 25mers, 45mers and 60mers. The results of these runs are set forth in FIGS. 10-12, where signal strength is indicated on the Y-axis and Run number is indicated on the X-axis. The boxes indicate different slides. As can be seen with reference to FIGS. 10-12, the signal strengths of runs 159, 161 and 163 are strong and compare well to the signals obtained from arrays fabricated by use of an anhydrous chamber alone.

Accordingly, the methods and devices of the invention offer a remarkable advance over conventional array fabrication methods in that they provide for the fabrication of biopolymer arrays without the use of an anhydrous chamber.

Although the foregoing embodiments in accordance with the invention have been described in some detail by way of illustration and example for purposes of clarity of understanding, certain changes and modifications may be made thereto without departing from the scope of the appended claims.

All publications and patents cited in this specification are herein incorporated by reference as if each individual publication or patent were specifically and individually indicated to be incorporated by reference.

While the invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the invention. All such modifications are intended to be within the scope of the claims appended hereto.

What is claimed is:

1. A method of depositing a reagent fluid onto a surface of a solid support, said method comprising:
a) vertically directing a gas onto a region of a surface of a solid support to produce a dried region; and
c) dispensing a reagent fluid onto said dried region.

2. The method according to claim 1, wherein said vertically directing said gas comprises flowing said gas onto said surface in a direction that is substantially normal to said surface.

3. The method according to claim 1, wherein said gas is vertically directed onto said surface from a gas jet.

4. The method according to claim 3, wherein said gas is flowing at a velocity of about 0.01 cm/s to about 1000 cm/s.

5. The method according to claim 1, wherein said reagent is dispensed onto said dried region from a pulse jet.

6. The method of claim 5, wherein said reagent fluid is dispensed from said pulse jet at a time ranging from about 0.01 s to about 1 s after said gas contacts said surface.

7. The method according to claim 1, wherein said reagent fluid is an activator, a solvent, a biopolymer or monomeric precursor of a biopolymer.

8. A pulse jet head assembly comprising a gas jet and a pulse jet.

9. The pulse jet head assembly according to claim 8, wherein said gas jet is adjacent to said pulse jet.

10. The pulse jet head assembly according to claim 8, wherein said assembly comprises a plurality of gas jets arranged in a row.

11. The pulse jet head assembly according to claim 10, wherein said assembly comprises a plurality of pulse jets arranged in a row that is adjacent to said row of gas jets.

12. The pulse jet head assembly according to claim 8, wherein said assembly comprises at least two gas jets separated by an intervening pulse jet.

13. The pulse jet head assembly according to claim 11, wherein said assembly comprises at least two rows of gas jets separated by a row of intervening pulse jets.

14. The pulse jet head assembly according to claim 13, wherein said pulse jet head assembly comprises N rows of pulse jets and N+1 rows of gas jets.

15. The pulse jet head according to claim 14, wherein said pulse jet head assembly comprises five or more rows of pulse jets.

16. The pulse jet head assembly according to claim 8, wherein at least one row of said five or more rows of pulse jets is operatively connected to a reservoir of a fluid activator.

17. The pulse jet head assembly according to claim 15, wherein at least one row of said five or more rows of pulse jets is operatively connected to a reservoir of a fluid nucleoside reagent.

18. The pulse jet head assembly according to claim 15, comprising at least one gas flow control element for actuating at least one of said gas jets.

19. A system for depositing a reagent fluid onto a surface of a solid support, said system comprising:
a) a station comprising a pulse jet head assembly for dispensing a plurality of fluids on to a surface of a solid support, wherein the pulse jet head assembly comprises a gas jet and a pulse jet;
b) one or more stations for contacting the surface of said solid support with another fluid; and
c) a positioning element for moving said solid support from one station to another.

20. A kit comprising the pulse jet head assembly of claim 8.
